(19) United States
(12) Patent Application Publication
(10) Pub. No.: US 2024/0138878 A1
(43) Pub. Date: May 2, 2024

(54) DEVICE AND METHOD TO REMOVE OBSTRUCTION FROM THROAT OF A USER

(71) Applicant: HCL Technologies Limited, New Delhi (IN)

(72) Inventor: PRASANNA DINESH N, Madurai (IN)

(21) Appl. No.: 18/385,019

(22) Filed: Oct. 30, 2023

(30) Foreign Application Priority Data
Oct. 31, 2022 (IN) 202211061827

(51) Int. Cl. A61B 17/50; A61B 17/24

(52) U.S. Cl. CPC A61B 17/50; A61B 17/24; A61B 2217/005

(57) ABSTRACT

The disclosure relates to devices and methods to remove obstruction from the throat of a user. The device includes a suction assembly including a housing and a plunger. The housing defines a housing proximal end and a housing distal end. The plunger defines a plunger proximal end and a plunger distal end, and is configured to be housed in and move within the housing between a first position and a second position. The device further includes a biasing member positioned between the plunger distal end and the housing distal end, and is configured to bias the plunger in the second position. The device further includes a locking assembly configured to hold the plunger in the first position and release the plunger upon user intervention.

### TECHNICAL FIELD

This disclosure relates generally to removing an obstruction from throat, and more particularly to system and method of removing (de-choking) an object lodged in the trachea of a user.

### BACKGROUND

Choking occurs when a piece of food or an object gets stuck in the throat or windpipe of a person. For example, during swallowing, a precise sequence of coordinated muscle movements guides food and liquids from the mouth into the esophagus. The epiglottis muscle is a coordinated muscle that acts as a barrier and prevents food from entering the trachea (windpipe). Inaccurate movement of the epiglottis muscle can cause food to get lodged in the trachea (windpipe). It blocks airflow in the trachea (windpipe), thereby resulting in asphyxiation or choking.

Choking can affect humans of all age groups. However, it is more common in infants and the elderly. An average response time for choking is 7 minutes, hence it is necessary to act quickly to address the choking in order to save the person from harm. The person who is choking may show symptoms like inability to breathe, speak, or cough. They may also have a bluish tint on the lips, skin, or nails from lack of Oxygen, in severe cases.

Some devices are available that are designed for removing the obstruction (for e.g. food) from the trachea by generating suction pressure. Such devices typically employ manual mechanism (for e.g. pull action) for generating suction pressure to remove the obstruction. However, the users are generally not aware about the required suction pressure. Therefore, such devices may not be suitable for user of all the age groups. For example, a higher suction pressure may harm an infant, and similarly, a lower suction pressure may be ineffective for adult users. Thus, existing anti-choking methods and anti-choking devices do not fully adhere to end customer's usability. Moreover, manufacturing anti-chocking devices specific to different age groups increased the cost of these devices.

### SUMMARY

In an embodiment, a device for removing an obstruction from throat of a user is disclosed. The device may include a suction assembly. The suction assembly may include a housing configured as a hollow cylindrical body and defining an inner surface and an outer surface. The housing may include a housing proximal end and a housing distal end. The device may further include a plunger configured to be housed and move within the housing. The plunger may define an outer surface. The plunger may include a plunger proximal end and a plunger distal end. The plunger may be configured to move within the hollow cylindrical body between a first position and a second position. In the first position, the plunger distal end may be positioned near the housing distal end and in the second position, the plunger distal end may be positioned farther from the housing distal end. The device may further include a biasing member positioned between the plunger distal end and the housing distal end. The biasing member may be configured to bias the plunger in the second position. The device may further include a locking assembly which may include a plurality of the protrusions positioned on the outer surface of the plunger. The locking assembly may further include an engaging member attached to the housing. The engaging member may be configurable between a hold position and a release position. In the hold position, the engaging member is engaged with one of the plurality of protrusions to hold the plunger distal end near the housing distal end. In the release position, the engaging member is disengaged from the one of the plurality of protrusions, to allow the plunger distal end to move farther from the housing distal end, to thereby create vacuum with the housing in a region between the plunger distal end and the housing distal end.

In another embodiment, a method of removing obstruction from throat of a user is disclosed. The method may include manipulating a plunger within a housing and against a biasing member to position the plunger in a first position. In the first position, a plunger distal end may be positioned near a housing distal end. The method may further include engaging an engaging member with one of a plurality of the protrusions, to hold the plunger distal end near the housing distal end. The plurality of protrusions may be positioned on the outer surface of the plunger. The method may further include disengaging the engaging member with one of the plurality of protrusions, to allow the plunger distal end to move farther from the housing distal end under effect of the biasing member, to create vacuum within the housing in a region between the plunger distal end and the housing distal end and thereby apply cause suction at the throat of the user. The method may further include positioning a face mask at the face of a user, to enclose mouth and nose of the user. The face mask may define an opening at its centre to allow a second end of a throat tube to pass through the opening associated with the face mask and enter into the mouth of the user. A first end of the throat tube may be connected to the housing distal end.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a perspective view of an exemplary device for removing an obstruction from throat of a user, in accordance with some embodiments of the present disclosure.

FIG. 2 illustrates an exploded view of the exemplary device of FIG. 1, in accordance with some embodiments of the present disclosure.

FIG. 3 is a process diagram of a process of using the device of FIG. 1 for removing obstruction from throat of a user corresponding to a first-level protrusion, in accordance with some embodiments.

FIG. 4 is a process diagram of a process of using the device of FIG. 1 for removing obstruction from throat of a user corresponding to a second-level protrusion, in accordance with some embodiments.

FIG. 5 illustrates a perspective view of the exemplary device of FIG. 1 positioned on a user for removing an obstruction from throat of the user, in accordance with some embodiments of the present disclosure.

FIG. 6 illustrates a flowchart of a method of removing obstruction from throat of a user using the device of FIG. 1, in accordance with some embodiment of the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

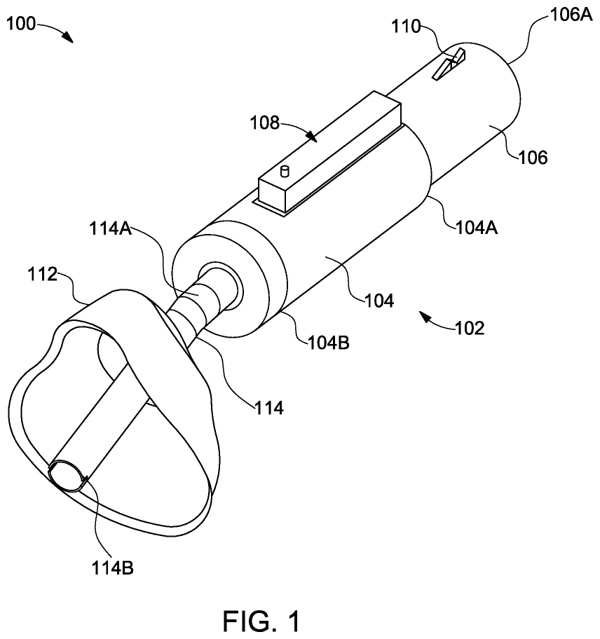

Referring now to FIG. 1, a perspective view of a device 100 for removing an obstruction from the throat of a user is disclosed is illustrated, in accordance with some embodiments of the present disclosure. The device 100 may provide a compact, lightweight, portable, and low-cost solution for removing an object lodged in the throat of a user. The device 100 may include a suction assembly 102 which may include a housing 104 configured as a hollow cylindrical body. The hollow cylindrical housing 104 may include a housing proximal end 104A and a housing distal end 104B. The suction assembly 102 may further include a plunger 106 configured to be housed and move within the housing 104. For example, the plunger 106 may be configured as an elongated solid cylindrical body defining an inner surface and an outer surface. The plunger 106 may include a plunger proximal end 106A and a plunger distal end (not shown in FIG. 1). The housing proximal end 104A may be open to receive the plunger distal end. The plunger 106 may be slidably disposed within the housing 104, such that the plunger 106 is moveable therein between a first position and a second position. At least a part of the plunger 106 may be sealed against the walls of the hollow cylindrical housing 104 to establish an air-tight contact with the inner surface of the housing. The plunger 106 may further include a sealing ring (not shown in FIG. 1) to form an air-tight contact with the inner surface of housing 104 in order to create and secure vacuum needed to create suction and draw the lodged object from the throat of the user.

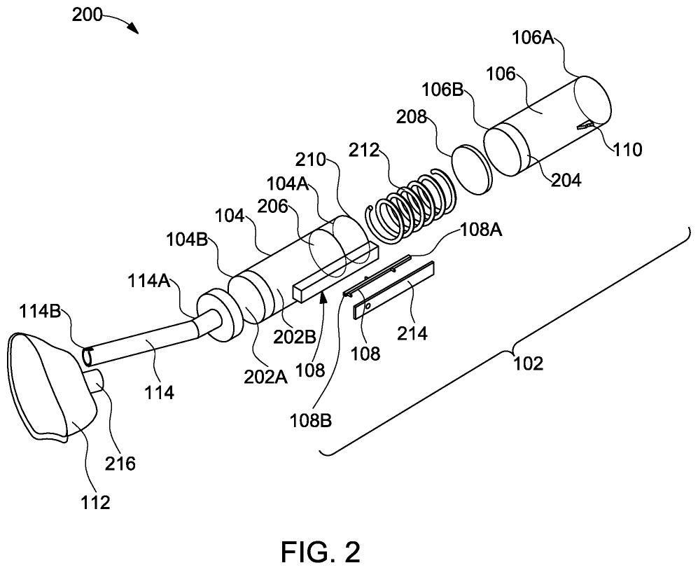

In the first position, the plunger distal end may be positioned near the housing distal end 104B. In the second position, the plunger distal end may be positioned farther from the housing distal end 104B. Further, the device 100 may include a biasing member (not shown in FIG. 1, refer FIG. 2) positioned between the plunger distal end and the housing distal end 104B and configured to bias the plunger 106 in the second position.

The device 100 may further include a locking assembly configured to either hold or release the plunger 106 in the second position. The locking assembly may include a plurality of protrusions 110 positioned on the outer surface of the plunger 106 and an engaging member 108 attached to the housing 104. The engaging member 108 may be configurable between a hold position and a release position. In the hold position, the engaging member 108 may be engaged with one of the plurality of protrusions 110 to hold the plunger distal end 106B near the housing distal end 104B (when the plunger is configured in the first position). In the release position, the engaging member 108 may be disengaged from the one of the plurality of protrusions 110, to thereby release the plunger 106 and allow the plunger distal end to move farther from the housing distal end 104B under the effect of the biasing member. The movement of the plunger distal end farther from the housing distal end 104B creates vacuum within the housing 104 in a region between the plunger distal end and the housing distal end 104B. This vacuum creates a suction action which may be used for removing the obstruction from the throat of the user.

Referring now to FIG. 2, an exploded view 200 of the device 100 for remove obstruction from throat of a user is illustrated, in accordance with some embodiments. As mentioned above, the device 100 may include the suction assembly 102. The suction assembly 102 may include the housing 104 having the housing proximal end 104A and the housing distal end 104B. The housing 104 may be configured as an elongated hollow cylindrical body defining an inner surface 202A and an outer surface 202B. The suction assembly 102 may further include the plunger 106 configured to be housed in and to move within the housing 104. The plunger 106 may include the plunger proximal end 106A and a plunger distal end 106B. The plunger 106 may be slidably disposed within the housing 104, such that the plunger 106 may be moveable therein between the first position and the second position.

At least a part of the plunger 106 may be sealed against the inner surface 202A of the hollow cylindrical housing 104 to form an air-tight contact with the inner surface 202A of the housing 104. To this end, the plunger 106 may include a sealing ring 204 to establish air-tight contact with the inner surface 202A of housing 104 in order to create the vacuum needed to suck in the lodged object from the throat of the user.

The housing 104 may further include a guide ring 206 to establish an air-tight contact with the outer surface of plunger 106 and to avoid direct contact of the outer surface of the plunger 106 with the inner surface 202A of the housing 104. The guide ring 206 may therefore reduce friction and ensure smooth movement of the plunger 106 inside the housing 104 while also acting as air seal. In some example embodiments, the guide ring 206 made of silicone material.

The housing 104 may further include a detent ring 208. The detent ring 208 may be configured to engage with the plunger distal end 106B of the plunger 106. The detent ring 208 may be positioned within the housing 104 and towards the housing proximal end 104A, to restrict the movement of the plunger 106 beyond it. As such, the detent ring 208 may prevent the movement of the plunger 106 beyond the second position, after the plunger 106 starts moving from the first position to the second position. To this end, additionally, the housing 104 may include a lip 210 formed at the housing proximal end 104A. The detent ring 208 may be stopped by the inwardly protruding lip 210 to thereby stop the movement of the plunger 106 beyond the housing proximal end 104A and stop the plunger 106 from falling out of the housing 104. In some embodiments, the detent ring 208 may be provided as a protrusion at the inner surface of the housing 104. In some alternate embodiments, the detent ring 208 may be removably attached to the inner surface of the housing 104. In some other alternate embodiments, the detent ring 208 may be attached to the plunger distal end 106B of the plunger 106.

The device 100 may further include a biasing member 212 positioned between the plunger distal end 106B and the housing distal end 104B. The biasing member 212 may be one of a compression spring, a flexible rubber member, a torsional spring, etc. For example, in some embodiments, a compression spring made of alloy steel may be used as the biasing member 212. The biasing member 212 may be configured to contract and retract based upon the movement of the plunger 106, upon manipulation by a user, as will explained in subsequent sections of the present disclosure. The biasing member 212 may be configured to bias the plunger 106 in the first position, i.e. when the plunger distal end 106B positioned near the housing distal end 104B.

The device 100 may further include the locking assembly which may include the engaging member 108 positioned on the housing 104 and the plurality of protrusions 110 provided on the plunger 106. The engaging member 108 may be configurable between the hold position and the release position. In the hold position, the engaging member 108 may be engaged with one of the plurality of protrusions 110 of the plunger 106 to hold the plunger distal end 106B near the housing distal end 104B. As such, in the first position of the plunger 106, the biasing member 212 (e.g. compression spring) may be compressed (against its normal state), and the second position may be retained by holding the plunger 106 using the engaging member 108 and one of the plurality of protrusions 110.

In order to release the plunger 106, the user has to manipulate the engaging member 108, to thereby disengage the engaging member 108 from the one of the plurality of protrusions 110. Once the plunger 106 is released, the biasing member 212 may cause the plunger 106 to move the from the first position to the second position. The movement of the plunger distal end 106B farther from the housing distal end 104B creates vacuum within the housing 104 in the region between the plunger distal end 106B and the housing distal end 104B.

It should be noted that the plunger 106 may be configured in the first position by pushing the plunger 106 (for example, by applying a force at the proximal end 106A) against the biasing member 212. The device 100, with the plunger 106 configured in the first position, may be positioned on the mouth and nose region of the user (i.e. a person who is suffering from a choked throat condition). Upon positioning, the plunger 106 may be released from the engaging member 108 by manipulating the engaging member 108, to cause the plunger 106 to move from the second position to the first position under the effect of the biasing member 212. As the plunger 106 moves from the first position to the second position, the plunger 106 create vacuum in the housing 104 and a corresponding suction in the throat of the user which may help in sucking in and removing the obstruction from the throat of the user.

As mentioned above, in the first position, the plunger distal end 106B may be positioned near the housing distal end 104B. Further, in the first position, the biasing member 212 may be in a compressed state. In the second position, the plunger distal end 106B may be positioned farther from the housing distal end 104B. Further, in the second position, the biasing member 212 may be in a substantially uncompressed state. The tendency of the biasing member 212 to assume the normal uncompressed state may cause the plunger distal end 106B to be pushed farther from the housing distal end 104B.

In some embodiments, the engaging member 108 may define a first end 108A and a second end 108B. The engaging member 108 may be configured as an elongated structure defining the first end 108A and the second end 108B. Further, for example, the first end 108A may be configured to engage with one of the plurality of protrusions 110 positioned on the plunger 106 in the hold position of the engaging member 108. The engaging member 108 may be operable by manipulating the second end 108B by the user, to cause the first end 108A to disengage from the one of the plurality of protrusions 110 positioned on the plunger 106.

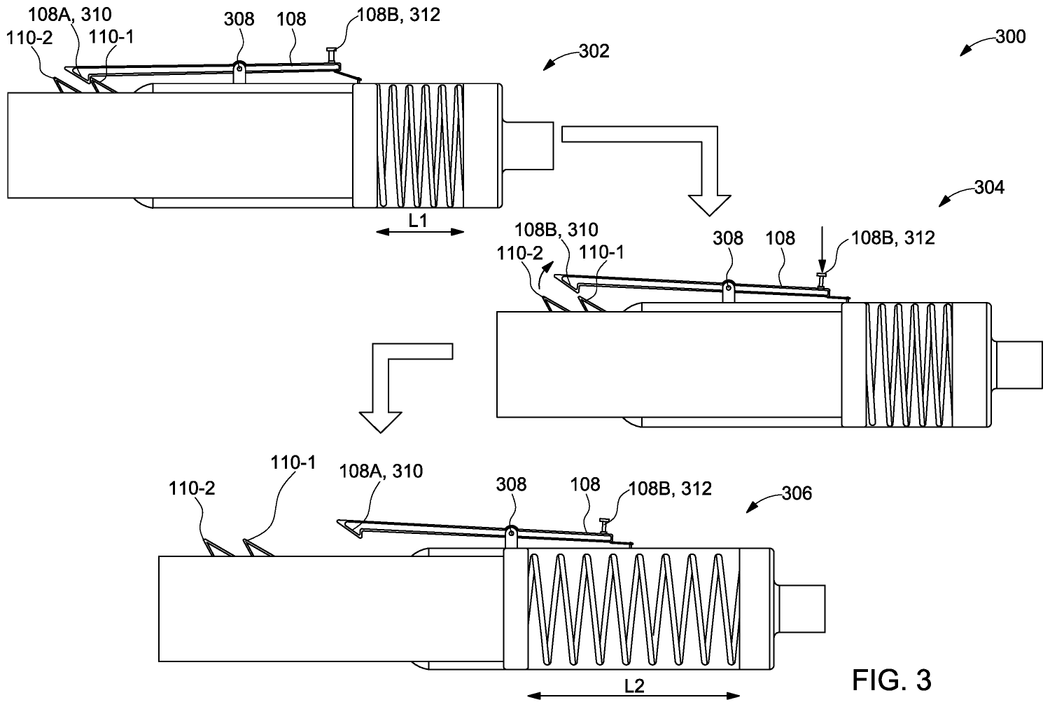

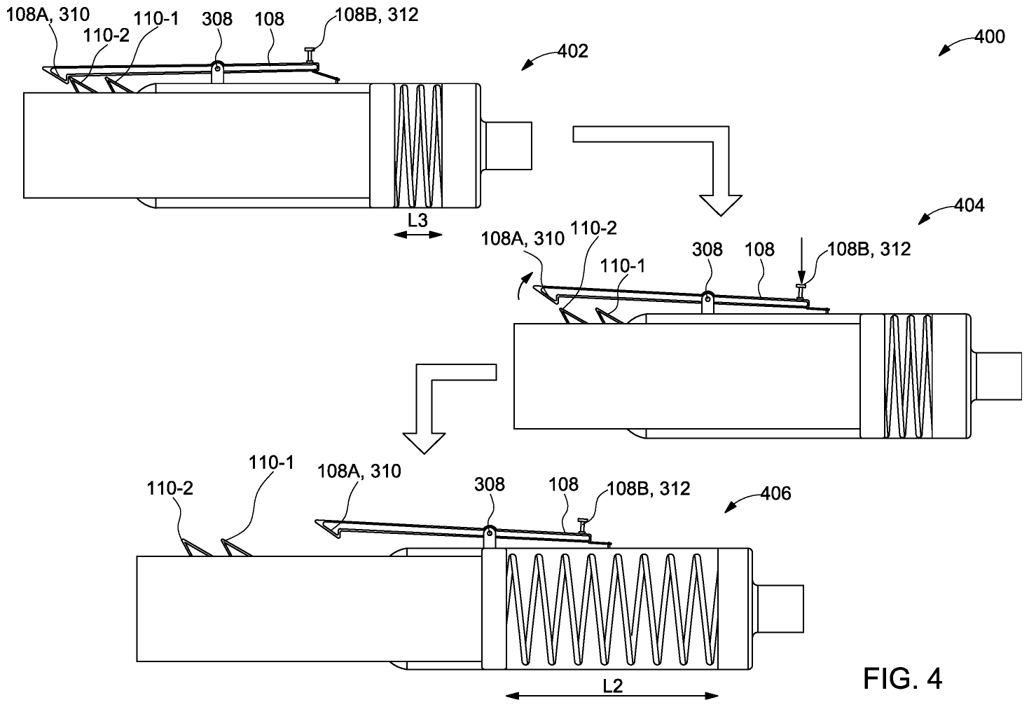

In one particular embodiment, the engaging member 108 may be configured as a lever pivoted at a pivot point (pivot point 308, refer, FIG. 3). Further, for example, the first end 108A (also referred to as first lever end 108A) may be configured to engage with one of the plurality of protrusions 110 positioned on the plunger 106, in the hold position of the engaging member 108. To this end, the first end 108A may include a hook (i.e. hook 310, refer FIG. 3), which may be configured to engage with one of the plurality of protrusions 110. The engaging member 108 may be operable by manually pressing (for example, using a finger-tip) the second end 108B (also referred to as second lever end 108A) to cause the engaging member 108 to rotate about the pivot point and assume the release position, to thereby disengage from the one of the plurality of protrusions 110 from the hook 310 of the engaging member 108. To this end, the second end 108B may include a push-button (i.e. push-button 312, refer FIG. 3) to allow the user to manipulate the engaging member 108 by manually pressing push-button at the second end 108B. The locking assembly is further explained in detail, in conjunction with FIG. 3-4.

In some embodiments, the locking assembly may further include a locking assembly cover 214 which may be configured to cover the components of the locking assembly. The locking assembly cover 214 may be an elongated member and configured to be positioned over the engaging member 108. As will be understood, the device 100, in some embodiments, may be sold with the plunger 106 being configured in the first position, as a ready-to-use product. As such, it may be important to protect the engaging member 108 from being manipulated accidently, as the accidental manipulation may cause the plunger 106 to be configured into the second position (hence, no longer ready-to-use).

It should be noted that some alternate embodiments of the locking assembly may be implemented as well, for achieving the above functionalities. For example, instead of the engaging member 108 configured as the lever, the engaging member 108 may be configured as a sliding member which may selectively engage or disengage with a protrusion of the plunger, during the sliding movement.

As already mentioned above, the device 100 may further include the face mask 112 which may define an opening 216 at its center. The face mask 112 may be a universal fitting face mask which may accommodate users of various different age groups. The device 100 may further include the throat tube 114 configured to attach to the housing distal end 104B, via hole defined at the housing distal end 104B. The throat tube 114 may define the first end 114A which may be attached to the housing distal end 104B. The throat tube 114 may further define the second end 114B which may be configured to pass through the opening 216 associated with the face mask 112 and enter into the mouth of the user.

Referring now to FIG. 3, a process diagram of a process 300 of using the device 100 for removing obstruction from throat of a user is illustrated, in accordance with some embodiments. The process 300 is explained hereby in conjunction with the device 100, as explained above in FIGS. 1-2.

At step 302, the plunger 106 of the device 100 is configured in the first position. As mentioned above, in the first position, the plunger distal end 106B may be positioned near the housing distal end 104B. In the first position, the biasing member 212 may be in compressed state. The plunger 106 may be configured in the first position by pushing the plunger 106 (for example, by applying a force at the proximal end 106A) against the biasing member 212. With the plunger 106 configured in the first position, the device 100 may be positioned on the mouth and nose region of the user (not shown in FIG. 3).

In the embodiment shown in FIG. 3, the engaging member 108 is configured as a lever pivoted at a pivot point 308. Further, the first end 108A of the engaging member is engaged with a first-level protrusion 110-1 of the plurality of protrusions 110 positioned on the plunger 106, in the hold position of the engaging member 108. As shown in FIG. 3, the plurality of protrusions 110 may include the first-level protrusion 110-1 and a second-level protrusion 110-2. Each of the first-level protrusion 110-1 and the second-level protrusion 110-2 may be positioned on the outer surface of the plunger 106. It should be noted that in the hold position, the engaging member 108 may be configured to be engaged with one of the first-level protrusion 110-1 and the second-level protrusion 110-2, to hold the plunger distal 106B end near the housing distal end 104B. As shown in FIG. 3, the engaging member 108 is engaged with the first-level protrusion 110-1, to thereby hold the plunger distal 106B end near the housing distal end 104B. In this embodiment, in the first position of the plunger 106, a separation between the plunger distal end 106B and the housing distal end 104B may be distance L1. Further, it should be noted that a suction pressure generated when the engaging member 108 is engaged with the first-level protrusion 110-1 may be in a range of 10 kilopascal (KPa) to 13 KPa. This suction pressure may be less than a suction pressure generated when the engaging member 108 is engaged with the second-level protrusion 110-2. As such, the suction pressure corresponding to the engaging member 108 being engaged with the first-level protrusion 110-1 may be suitable for kids and teenagers.

At step 304, the plunger 106 of the device 100 is configured in an intermediary position. In other words, at step 304, the engaging member 108 is disengaged from the first-level protrusion 110-1, i.e. the engaging member 108 is removed from the hold position, for example, by manipulating (by the user) the engaging member 108. The user may press a push-button 312 at the second end 108B to cause the engaging member 108 to rotate about the pivot point 308 and assume the release position. In the release position, a hook 310 at the first end 108A of the engaging member 108 may disengage from the first-level protrusion 110-1 of the plunger 106. Once the first end 108A (i.e. the hook 310) of the engaging member 108 is disengaged from the first-level protrusion 110-1, the plunger 106 is released and may start moving from the first position towards the second position under the effect of the biasing member 212. As the plunger 106 moves from the first position to the second position, the plunger 106 creates vacuum in the housing 104 and a corresponding suction in the throat of the user which may help in sucking in and removing the obstruction from the throat of the user. As a result, the separation between the plunger distal end 106B and the housing distal end 104B may start increasing (beyond L1).

At step 306, the plunger 106 of the device 100 is configured in the second position. After the first end 108A (i.e. the hook 310) of the engaging member 108 is disengaged from the first-level protrusion 110-1, the plunger 106 may be eventually configured in the second position. In the second position, the plunger distal end 106B is positioned farther from the housing distal end 104B. As a result, the separation between the plunger distal end 106B and the housing distal end 104B, at step 306, is distance L2. Once the plunger 106 reaches the second position, the detent ring 208 may engage with the lip 210 of the housing 104, and the lip 210 may stop the movement of the plunger beyond the housing proximal end 104A and prevent the plunger 106 from falling outside the housing 104. In the second position of the plunger 106, the separation (i.e. the distance L2) between the plunger distal end 106B and the housing distal end 104B is the maximum possible separation between the plunger distal end 106B and the housing distal end 104B. Therefore, during the transition from the first position to the second position, the plunger 106 covers a first travel distance of L2-L1.

Further, at step 306, the suction action is complete. As will be understood, if the suction action is successful in removing the obstruction from the throat of the user, the device 100 may be removed from the mouth and nose region of the user. However, if the suction action is not successful in removing the obstruction from the throat of the user, the device 100 may be used again to re-perform the suction action. To this end, the plunger 106 may be manually configured in the first position by the user (i.e. the user on which the device 100 is being user) himself or by another user. As such, the steps 302 to 306 may be repeated once again.

Referring now to FIG. 4, a process diagram of a process 400 of using the device 100 for removing obstruction from throat of a user is illustrated, in accordance with another embodiment. The process 400 is explained hereby in conjunction with the device 100, as explained above in FIGS. 1-2.

At step 402, the plunger 106 of the device 100 is configured in the first position, i.e. the plunger distal end 106B may be positioned near the housing distal end 104B, and the biasing member 212 may be in compressed state. The plunger 106 may be configured in the first position by pushing the plunger 106 against the biasing member 212. With the plunger 106 configured in the first position, the device 100 may be positioned on the mouth and nose region of the user (not shown in FIG. 4). In the embodiment shown in FIG. 4, the engaging member 108 is configured as a lever pivoted at a pivot point 308. Further, the first end 108A of the engaging member is engaged with the second-level protrusion 110-2 of the plurality of protrusions 110 positioned on the plunger 106, in the hold position of the engaging member 108. The second-level protrusion 110-2 may be positioned on the outer surface of the plunger 106. In the hold position, the engaging member 108 may be engaged with the second-level protrusion 110-2, to hold the plunger distal 106B end near the housing distal end 104B, as shown in FIG. 4. In this embodiment, in the first position of the plunger 106, a separation between the plunger distal end 106B and the housing distal end 104B may be distance L3. Further, it should be noted that a suction pressure generated when the engaging member 108 is engaged with the second-level protrusion 110-2 may be in a range of 13 KPa to 16 KPa. As such, this suction pressure may be suitable for adults.

At step 404, the plunger 106 of the device 100 is configured in an intermediary position. In other words, at step 404, the engaging member 108 is disengaged from the second-level protrusion 110-2, i.e. the engaging member 108 is removed from the hold position, for example, by manipulating by the user. The user may press the second end 108B to cause the engaging member 108 to rotate about the pivot point 308 and assume the release position, to thereby disengage the first end 108A (i.e. the hook 310) from the second-level protrusion 110-2 of the plunger 106. Once the first end 108A of the engaging member 108 is disengaged from the second-level protrusion 110-2, the plunger 106 is released and may start moving from the first position towards the second position under the effect of the biasing member 212. As the plunger 106 moves from the first position to the second position, the plunger 106 may create vacuum in the housing 104 and a corresponding suction in the throat of the user which may help in sucking in and removing the obstruction from the throat of the user. As a result, the separation between the plunger distal end 106B and the housing distal end 104B may start increasing (beyond L3).

At step 406, the plunger 106 of the device 100 is configured in the second position. After the first end 108A of the engaging member 108 is disengaged from the second-level protrusion 110-2, the plunger 106 may be eventually configured in the second position. In the second position, the plunger distal end 106B is positioned farther from the housing distal end 104B. As a result, the separation between the plunger distal end 106B and the housing distal end 104B, at step 306, is distance L2. Once the plunger 106 reaches the second position, the detent ring 208 may engage with the lip 210 of the housing 104, and the lip 210 may stop the movement of the plunger 106 beyond the housing proximal end 104A and prevent the plunger 106 from falling outside the housing 104. In the second position of the plunger 106, the separation (i.e. the distance L2) between the plunger distal end 106B and the housing distal end 104B is the maximum possible separation between the plunger distal end 106B and the housing distal end 104B. Therefore, during the transition from the first position to the second position, the plunger 106 covers a second travel distance of L2-L3. At step 406, suction action is complete.

It should be noted that the second travel distance L2-L3 (corresponding to the second-level protrusion 110-2 may be greater than first travel distance L2-L1 corresponding to the first-level protrusion 110-1. As such, the amount of suction created by the plunger 106 during covering of the second travel distance L2-L3 may be greater than the amount of suction created by the plunger 106 during covering of the first travel distance L2-L1.

The device 100 is able to generate a precise suction pressure as plunger 106 retracts automatically on pressing of the push-button 312. The precise suction pressure may be adjusted according to the age of the user who is suffering from the choking condition. In some embodiments, the required suction pressure in order to remove a lodged object from the throat of a teen or an infant is in the range of 10 KPa to 13 KPa, and the required suction pressure in order to remove a lodged object from the throat of an adult is in the range of 13 KPa to 16 KPa. The plurality of protrusions 110 provided on the plunger 106 provide for the precise suction pressure according to the age of the choked person, as each of the plurality of protrusions 110 determine the spring compression force and eventually the suction pressure to be produced.

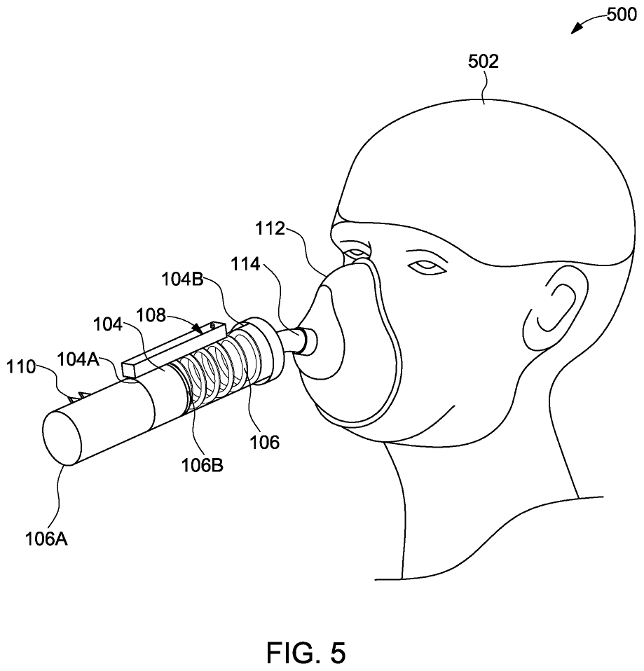

Referring now to FIG. 5 a perspective view 500 of an application of device 100 for removing an obstruction from throat of a user 502 is illustrated, in accordance with some embodiments. The face mask 112 of the device 100 may be positioned over the mouth and nose region of the user 502, and the throat tube 11 may be inserted into the throat via the mouth of the user 502. The face mask 112 may be positioned so that the mouth and nose of the user 502 is completely enclosed therewithin. In some embodiments, the device 100 may be used with the plunger 106 already configured in the first position. Alternately, the device 100 may be first positioned on the mouth and nose region of the user 502 and then the plunger 106 may be configured in the first position by pushing the plunger distal end 106B against the biasing member 212. Upon positioning the face mask 112 and the throat tube 114, the engaging member 108 may be manipulated by the user 502 (or another person), to thereby release the plunger 106 and cause the plunger to move into the second position thereby creating vacuum and suction which may dislodge the object lodged in the throat of the user.

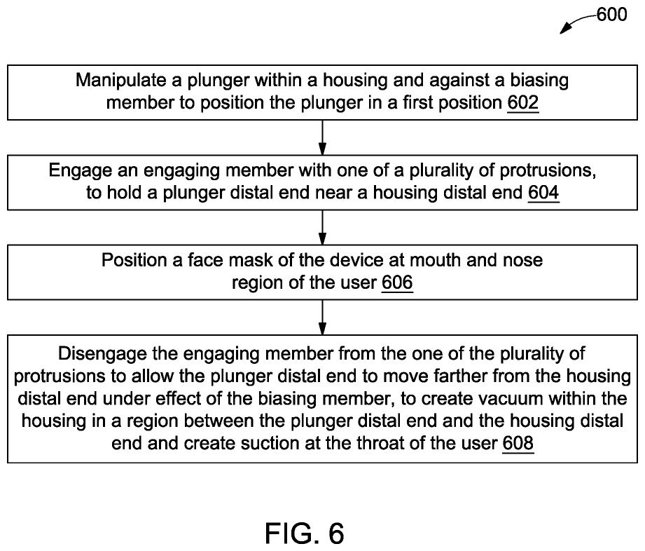

Referring now to FIG. 6, a flowchart of a method 600 of removing obstruction from throat of a user using the device 100 is illustrated, in accordance with some embodiments. At step 602, the plunger 106 of the device 100 may be manipulated within a housing and against the biasing member 212 to position the plunger 106 in the first position.

At step 604, the engaging member 108 may be engaged with one of the plurality of protrusions 110 provided on the plunger 106, to hold the plunger distal end 106B near the housing distal end 104B. In some embodiments, the engaging member 108 may include a lever pivoted to the outer surface of the housing 104. The lever may include the first lever end 108A and second lever end 108B. The first lever end 108A may be configured to engage with one of the plurality of protrusions 110 positioned on the outer surface of the plunger 106. The second lever end 108B may be configured to be manipulated by a user to thereby disengage the engaging member 108 from the one of the plurality of protrusions 110. It should be noted that engaging may include engaging member 108 with one of the first-level protrusion 110-1 and the second-level protrusion 110-2. The suction pressure generated when the engaging member 108 is engaged with the first-level protrusion 110-1 may be in the range of 10 KPa to 13 KPa. The suction pressure generated when the engaging member 108 is engaged with the second-level protrusion 110-2 may be in the range of 13 KPa to 16 KPa.

At step 606, the face mask 112 of the device 100 may be positioned at mouth and nose region of the user. The face mask 112 may define the opening 216 to allow the second end 114B of the throat tube 114 to pass through the opening 216 and enter into the mouth of the user. The first end of the throat tube 114A may be connected to the housing distal end 104B.

At step 608, the engaging member 108 may be disengaged from the one of the plurality of protrusions 110 to allow the plunger distal end 106B to move farther from the housing distal end 104B under effect of the biasing member 212, to create vacuum within the housing 104 in the region between the plunger distal end 106B and the housing distal end 104B and create suction at the throat of the user.

The above subject matter discloses devices and methods for safely and effectively removing obstruction from the throat of a user. The above device provides for a lightweight, compact, and easy-to-use solution for the anti-choking applications. No critical training is required to use the device, and device may be used by anyone regardless of their physical strength. Further, the device can be easily operated by the choked person, thereby removing dependency on another user for using the device. The above device doesn't require the plunger to be pulled manually at the time of use; instead, the plunger is automatically retracted with the help of a simple push button mechanism under the effect of a biasing member. Furthermore, the device provides for precise suction pressure for users of different age groups. Also, it is possible to adjust the pressure according to the age of the choked person, using different protrusions of the plurality of protrusions. For example, the device includes two protrusions—a first protrusion corresponding to relatively lower suction pressure and more suitable for infants, and a second protrusion corresponding to relatively higher suction pressure and more suitable for adults. Therefore, the device overcomes the requirement for manufacturing different masks for users of different age groups. Moreover, the device includes a universal fitting mask which reduces the multiple count of masks required for users of different age groups.

It is intended that the disclosure and examples be considered as exemplary only, with a true scope and spirit of disclosed embodiments being indicated by the following claims.

What is claimed is:

1. A device to remove obstruction from throat of a user, comprising:
a suction assembly, comprising:
a housing configured as a hollow cylindrical body and defining an inner surface and an outer surface, wherein the housing comprises:
a housing proximal end; and
a housing distal end;
a plunger configured to be housed and move within the housing, the plunger defining an outer surface, wherein the plunger comprises:
a plunger proximal end; and
a plunger distal end;
wherein the plunger is configured to move within the hollow cylindrical body between a first position and a second position;
wherein in the first position, the plunger distal end is positioned near the housing distal end; and
wherein in the second position, the plunger distal end is positioned farther from the housing distal end;
a biasing member positioned between the plunger distal end and the housing distal end and configured to bias the plunger in the second position; and
a locking assembly, comprising:
a plurality of protrusions positioned on the outer surface of the plunger; and
an engaging member attached to the housing, wherein the engaging member is configurable between a hold position and a release position;
wherein in the hold position, the engaging member is engaged with one of the plurality of protrusions to hold the plunger distal end near the housing distal end; and
wherein in the release position, the engaging member is disengaged from the one of the plurality of protrusions, to allow the plunger distal end to move farther from the housing distal end, to thereby create vacuum with the housing in a region between the plunger distal end and the housing distal end.

2. The device as claimed in claim 1, wherein the biasing member is one of: a compression spring, a flexible rubber member, and a torsional spring.

3. The device as claimed in claim 1, wherein the engaging member comprises:
a lever pivoted to the outer surface of the housing and comprising:
a first lever end; and
a second lever end;
wherein the first lever end is configured to engage with one of the plurality of protrusions positioned on the outer surface of the plunger, and
wherein the second lever end is configured to be manipulated by a user to thereby disengage the engaging member from the one of the plurality of protrusions.

4. The device as claimed in claim 1, wherein at least a part of the plunger forms an air-tight contact with the inner surface of the housing.

5. The device as claimed in claim 1 further comprising:
a face mask defining an opening at its centre, wherein the face mask is configured to be positioned at the face of a user, to enclose mouth and nose of the user.

6. The device as claimed in claim 1,
wherein the housing distal end defines a hole; and
wherein the housing distal end further comprises:
a throat tube defining:
a first end connected to the housing distal end, via the hole; and
a second end, wherein the second end of the throat tube is configured to pass through an opening associated with a face mask and enter into mouth of a user.

7. The device as claimed in claim 1, wherein the locking assembly comprises:
a first-level protrusion; and
a second-level protrusion;
wherein each of the first-level protrusion and the second-level protrusion, is positioned on the outer surface of the plunger,
wherein in the hold position, the engaging member is configured to be engaged with one of the first-level protrusion and the second-level protrusion, to hold the plunger distal end near the housing distal end,
wherein suction pressure generated when the engaging member is engaged with the first-level protrusion is in a range of 10 kilopascal (KPa) to 13 KPa, and
wherein suction pressure generated when the engaging member is engaged with the second-level protrusion is in a range of 13 KPa to 16 KPa.

8. A method of removing obstruction from throat of a user, comprising:
manipulating a plunger within a housing and against a biasing member to position the plunger in a first position,
wherein in the first position, a plunger distal end is positioned near a housing distal end; and
engaging an engaging member with one of a plurality of protrusions, to hold the plunger distal end near the housing distal end,
wherein the plurality of protrusions are positioned on an outer surface of the plunger; and
disengaging the engaging member with one of the plurality of protrusions, to allow the plunger distal end to move farther from the housing distal end under effect of the biasing member, to create vacuum within the housing in a region between the plunger distal end and the housing distal end and thereby apply cause suction at the throat of the user.

9. The method as claimed in claim 8 further comprising:
positioning a face mask at the face of a user, to enclose mouth and nose of the user,
wherein the face mask defines an opening at its centre to allow a second end of a throat tube to pass through the opening associated with the face mask and enter into the mouth of the user, and
wherein a first end of the throat tube is connected to the housing distal end.

10. The method as claimed in claim 8, wherein the engaging comprises:
engaging the engaging member with one of a first-level protrusion and a second-level protrusion,
wherein suction pressure generated when the engaging member is engaged with the first-level protrusion is in a range of 10 KPa to 13 KPa, and
wherein suction pressure generated when the engaging member is engaged with the second-level protrusion is in a range of 13 KPa to 16 KPa.
